(19) United States
(12) Patent Application Publication
(10) Pub. No.: US 2002/0103494 A1
(43) Pub. Date: Aug. 1, 2002

(54) Percutaneous cannula delvery system for hernia patch

(76) Inventor: John Allen Pacey, Vancouver (CA)

Correspondence Address: DR JOHN A. PACEY, 6290 COLLINGWOOD ST, VANCOUVER, BC, V6N 1T6, CA

(21) Appl. No.: 10/058,269

(22) Filed: Jan. 30, 2002

Related U.S. Application Data
(60) Provisional application No. 60/264,999, filed on Jan. 31, 2001.

(51) Int. Cl. A61B 17/08

(52) U.S. Cl. 606/151

(57) ABSTRACT

A method and prosthesis design and manufacture is described for repair of Inguinal and other Hernias by a combination of a percutaneous endoscopic imaging system with a proprietary patch. The nitinol or metal reinforced plastic fabric patch is introduced into the hernial defect by use of a cannula and after expansion and deployment is capable of stabilizing the peritoneum by use of a unique radiating skeleton with polydirectional hooks and also provide the necessary structural support. The system may be enhanced by use of a harpoon toggle suture, which is an integral component of the system and provides unprecedented strength of the repair. The patch is constructed of an adhesion resistant layer, a nitinol, plastic or metal frame and also a layer or elements designed to promote adherence. An alternate unitary construction is contemplated made of molded plastic to reproduce all of the structural features important to the function of the system.

### FIELD OF INVENTION

Hernia repair surgery was done for many years by means of direct suturing techniques. The techniques often failed because the local tissues lacked strength so mesh reinforcement strategies were developed. The technique has evolved over recent years to involve placement of mesh plugs , mesh implants. Cannula delivery has been described. Many procedures are now done with local or regional anesthetic techniques. ePTFE (expanded polytetrafluroethylene or Goretex®) has been used in a hernia patch placed in the peritoneum with laparoscopy and stapled with metal staples as a simple onlay but was found to be inferior to preperitoneal techniques as it was associated with a higher failure rate. Laparoscopic repair is also done widely using a preperitoneal mesh implant for strength. This solution is unique in combining a hooked reinforced intraperitoneal or preperitoneal patch made from polytetrafluroethylene, silicon, or some non adherent material.

### BRIEF DESCRIPTION OF THE INVENTION

The development here uses local anesthesia, a percutaneous trocar and cannula delivery method that may be done rapidly and in radiology suites as well as in surgical operating rooms. The use of laparoscopy imaging greatly facilitates the accurate placement of a blocking plug and reduces the time required to carry out the procedure. Local anesthesia is required.

The development of superelastic nitinol metal or other materials permit a strong moderate size patch to be placed through a cannula and have the predetermined shape recover completely from material memory after placement. Standard steel wire has less elastic recovery. Expanded PTFE graft materials have resulted in products that may be safely placed in the peritoneal cavity without fear of generation of excessive numbers of adhesive bands or adherence to the bowel. Thus material development has aided in creation of this unique solution to the hernia problem. The current filing defines additions to the preferred method of application of this technology.

Another addition to the concept introduces a layer of patch material that is adherent to the PTFE but has extra tissue adhesive qualities which enhance the adhesiveness of the device to the peritoneum and thus creates a more secure fixation. The addition of a permanent anchor for the device is also described. The addition of a method of manufacturing a complex molded patch with all elements formed with similar plastic, usually Teflon, eases production of the the prosthesis and reduces cost.

Another method requires the embedding of the frame and mechanical elements into silicone of medical grade.

### DETAILED DESCRIPTION OF PREFERRED EMBODIMENTS

The prosthesis may be created in mix of materials or in a solid molded or bonded plastic material or to avoid the use of metals. Material selection for a molded version should be suited to use mold. If the entire prosthesis is created in a plastic mold or of a composite construction with metals it should be done in such a way that union of all engineered parts is assured in the manufacturing process and the hooks , arms, webbing and anchor are all crafted to at once to anchor, prevent prosthesis movement , prevent peritoneal mobility, block the internal ring, and at the same time have flexibility characteristics similar to the host tissue so that some accommodation of the device occurs as required to prevent sensation in the implant area. Efforts to anchor the prosthesis are essential to prevent a migrating prosthesis which would have un-predictable consequences for the viscera. Our research has shown that the migration or sliding of peritoneum is a major factor in hernia size growth and also in development of recurrent hernia. This research is unique and was carried out during laparoscopic hernia operations wherein the mobility could be visualized with traction and changes in gas pressure.

Thus two strategies exist: the first using medical grade metal and plastic materials that are bonded together in such a way as to provide the same characteristics as the second all plastic device.

The composite method of construction involves the union of Nitinol superelastic alloy or other similar metal frame with ePTFE to produce a patch with strength and flexibility which will minimize the formation of intra abdominal adhesions. This bonding of the fabric and metal will result in a delivery size of 4-10 mm diameter but the patch will enlarge to a size of 40-100 mm when opened to a planar shape inside the abdomen. The nitinol hooked arms may be radial in pattern and have a design enabling support to be given to the ePTFE patch. The patch complex may be concave as is required in the human clinical application. The concave shape is preferred for Inguinal hernia repair to promote incorporation of tissue into the patch and to apply the patch to the internal hernia ring depression.

The nitinol arms in this preferred embodiment shall be fitted with hooks which will strongly resist migration or displacement of the device. The hooks on the radial arms will be directed both inward toward the patch center and also outward toward the edges so that a number of different angles of force will be applied with the effect that the peritoneum is stabilized and prevented from migrating toward the defect thus taking the patch with it.

The nitinol frame will be heat treated in the deployed configuration so that the memory of the metal will force the patch into optimal therapeutic shape when discharged from the carrier into the peritoneal space. The patch will then cover of the hernia defect with some overlap. ePTFE has been shown to produce few adhesions to bowel and omentum and will be more efficient in this regard because of the absence of any irregularity of the exposed surface of the ePTFE patch. The surface of the patch in direct contact with the abdominal wall peritoneum shall adhere to this tissue because that part of the prosthesis having an adherent nature is interactive with the patients tissues and so will form a permanent bond. This material will either be different from ePTFE ,which does not bond well to tissue or else have an ePTFE velour texture.

Many diverse delivery systems are conceptually possible to assist delivery of this prosthesis. The device may be introduced directly in an open abdominal operation or may be introduced through any delivery trocar of adequate size. The fixation method would differ in each case.

The following steps are required under regional anesthetic: Step 1 Pneumoperitoneum is established either via a Veres type needle, a Storz optical Veres needle or a Hassan type exposure in the lower quadrant of the abdomen on the side of the hernia with the patient in the slightly head down hernia side up position. Step 2 Place a 5 mm cannula and laparoscope. Step 3 Pass the patch delivery cannula and trocar directly through skin, subcutaneous fat, layers of fascia, and then directly through the peritoneum into the center of the hernia defect. The trocar is removed from the cannula and the ePTFE patch is introduced into the cannula and deployed by use of a pusher that retains the CO2 pneumoperitoneum in the abdomen. The device is aimed at the center point of the hernial defect depression at which point it springs open and the cannula may be removed. The traction is then applied to the tether attached to the patch. The laparoscope is again used to view the final result with aid of the modest volume pneumoperitoneum and the patient in a position allowing the viscera of the abdomen to fall with gravity. The patch may be adjusted with the cannula in place and when satisfactory will fill the inner ring with one end of the patch hooked on the inferior epigastric vessel leash which provides a strong point. The tether can be pulled snugly and fixed to complete the repair. Then the 10 mm skin wound may be taped or sutured as required.

The method has been largely described above but ingenuity will develop more varied methods for using the patch system. The main advantage of this method will be the very small incisions required, the ease of application and immediate recovery.

One of the variations that may be necessary is to incise peritoneum at mouth of sac as much as possible to transect the sac and leave the sac in place.

The innovation herein created is based upon the integration of simplified laparoscopic method with the direct delivery of the patch through the weakened area of the hernia via a cannula. The patch created is also unique , it has a form strengthened by radial arms which have hooked features to engage and stabilize the peritoneum. This concept is a result of the recognition of the importance of the sliding of the peritoneum into the hernial sac as a key cause of hernia growth and recurrence.

This system must also be distinguished from atrial septal defect style patches which are 2 part systems that are joined together on right and left sides of an intracardiac defect. These devices have no hooking features and rely on the central mechanical union of 2 parts for fixation. No other hernia system yet created provides such convenience of delivery, absence of pain, and immediate return to customary activity as the system described here.

The plastic used must be nonreactive to prevent adhesion formation and an alternate material may be silicon sheet bonded to prolene mesh and the hooked elements if this is determined to be a preferred strategy.

### BRIEF DESCRIPTION OF DRAWINGS

FIG. 1 Saggital view of insertion of the patch into the abdomen

FIG. 2 ePTFE patch planar surface adjacent to the viscera.

FIG. 3 ePTFE patch with hooked radial reinforcement and attached tissue adherent layer.

FIG. 4 Cross section view of composite materials version.

FIG. 5 Cross section view of the composite version with Anatomy showing the harpoon tacker application.

FIG. 6 Cannula with patch and pusher.

FIG. 7 Plastic patch in position from the laparoscopic view.

FIG. 8 Harpoon suture

a. Attached for delivery.

b. Detached in the ePTFE Patch preformed holes.

### DETAILED DESCRIPTION OF DRAWINGS

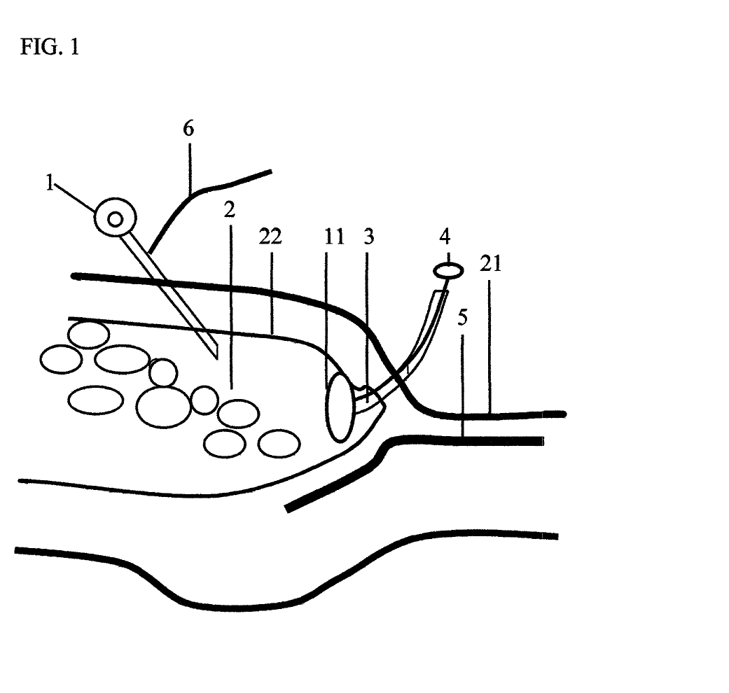

The general overview of the placement of the device in the body of a human subject may be seen in FIG. 1 where the process is viewed with a laparoscope 1 with the aid of a small pneumo-peritoneum delivered by the access channel 6. The bowel 2 is displaced by gravity away from the ePTFE patch 11 and the body penetration is made by the cannula 3 usually using a standard trocar (not seen). The site of penetration of the cannula through the skin 21 is directed through the peritoneum 22 toward the center of the hernia defect visualized by the laparoscope. This is done with care to avoid the femoral vessels 5 and also the inferior epigastric vessels (not seen).

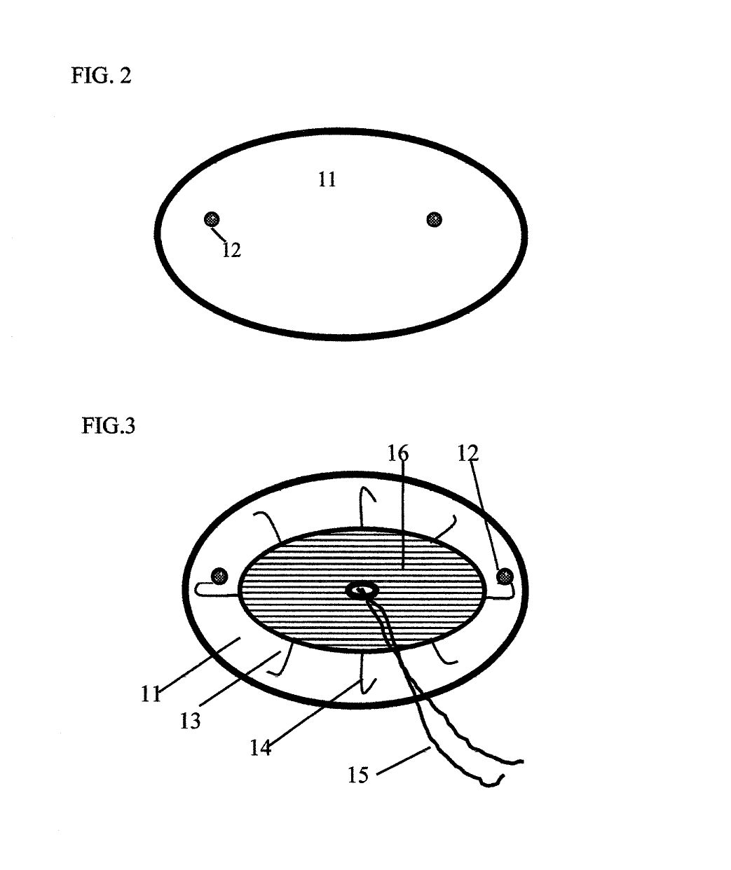

The ePTFE patch is shown in FIG. 2 from the side viewed by the laparoscope and exposed to the bowel and omentum. It is interchangeable for the right and left as it is symmetrical with two optional 2 mm. penetrations 12 for use of the Teflon® harpoon fixation system described later. The surface is otherwise free of defects or other irregularities that might provoke adhesions.

The opposite surface of the ePTFE patch 11 is applied to the peritoneal surface of the patient FIG. 3 and the holes 12 may be seen lateral to the adherence provoking layer 16 which is attached to the ePTFE and provides a permanent fixation of the complex to the abdominal wall. Two types of hooks 13 and 14 are present on structural arms which are united at the center of the patch and radiate outward and attach by the multidirectional hooks to the peritoneum. These hooks are directed alternately inward toward the center of the patch 14 and outward 13. The third and final means of fixation is by the tether 15 which is used to fix the structure to the muscle and fascia.

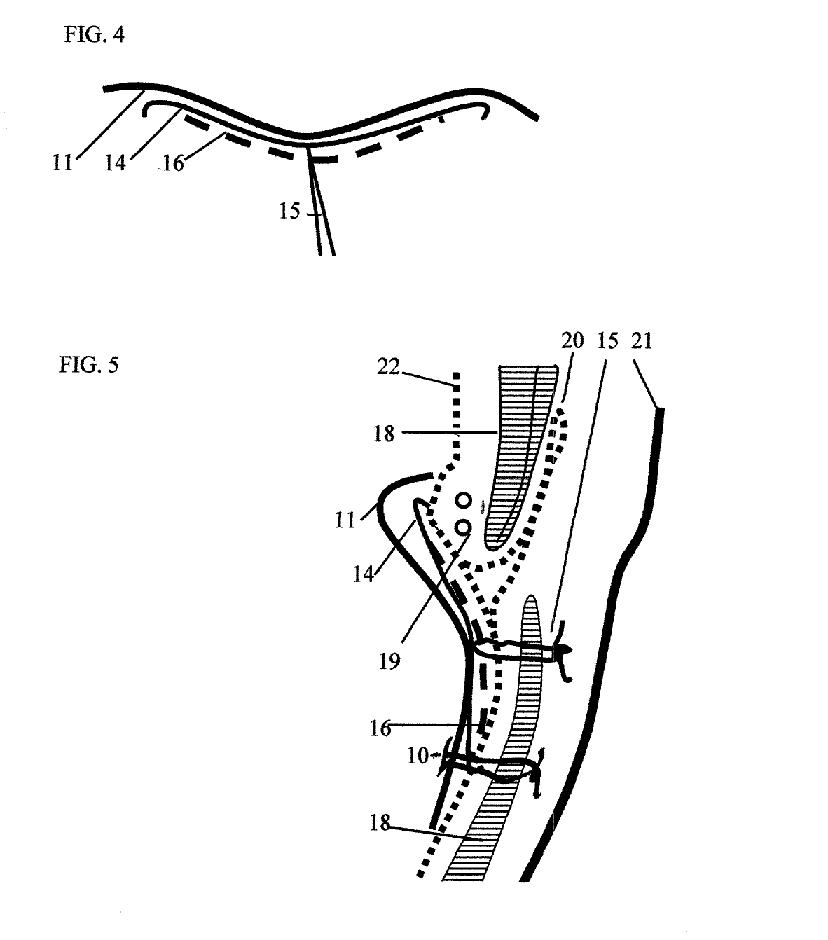

The profile view FIG. 4 shows other relationships the inner ePTFE layer 11 adjacent to the radiating metal or plastic arms 14 and the outermost layer 16 which is a material such as a velour that will tend to attach to peritoneum. The centrally attached tether is also shown 15.

The profile view in FIG. 4 is transferred to the groin area FIG. 5 and shows the placement of the elements with respect to the anatomy of the area. The musclo-fascial area 18 and the strong feature 19 the inferior epigastric vessels provide the strong support needed for the patch 11 and its arms. The bi-directional hooks 13 and 14 (only 14 is shown in this view), hook into the peritoneum 22 and are also supported by 16 the adherent layer attaching to the sac 20 area. The tether can be seen tied to the musculofascial area and the healed skin 21 is seen more superficially.

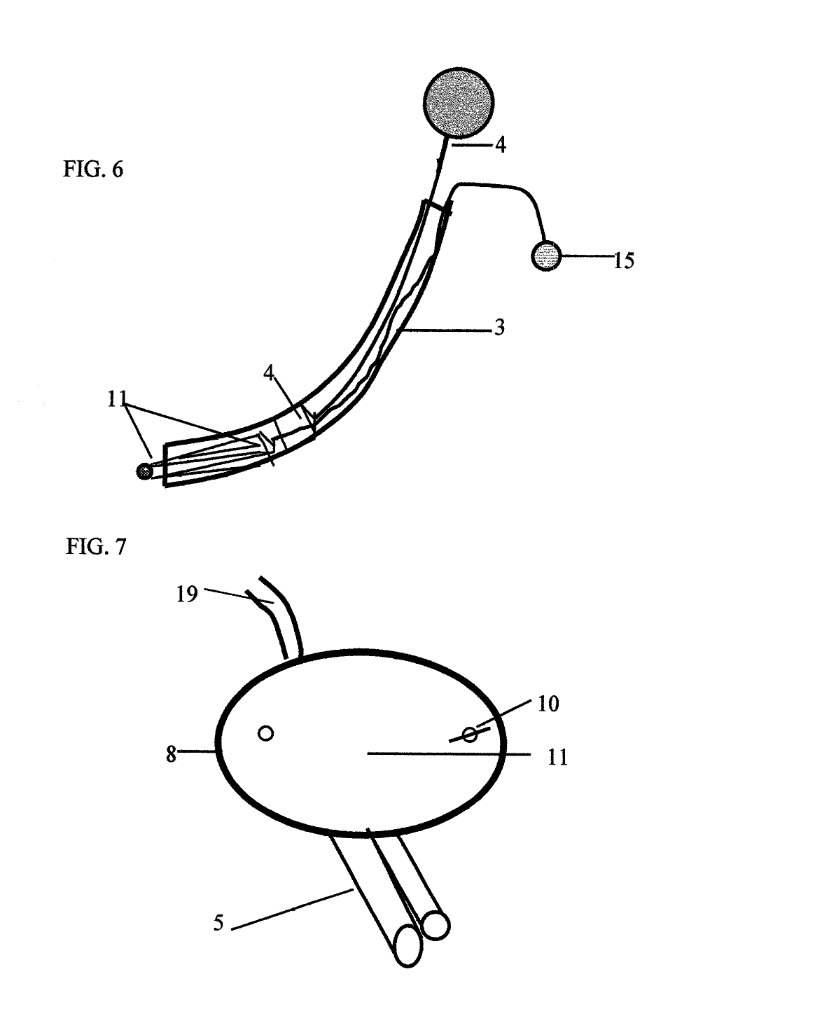

The cannula 3 and pusher 4 with its gas preserving piston are shown in FIG. 6. The folded ePTFE patch 11 is contained in the end. The tether 15 is held outside of the abdomen so that it might be tied to the musculofascial layer.

The view in FIG. 7 shows the medial side 8 of the ePTFE patch 11 overlying the inferior epigastric vessels 19 and the femoral vessels 5. The lateral edge of the patch is fixed to the abdominal wall with the head 10 of the harpoon fixation system.

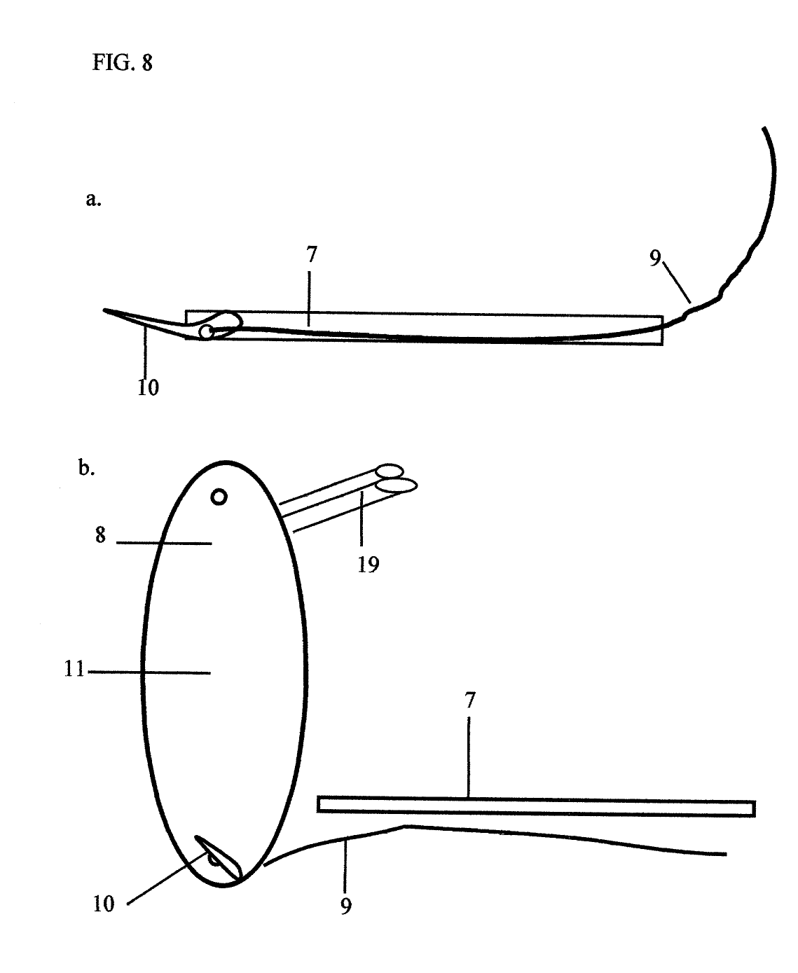

The detail of the harpoon fixation system is shown in FIG. 8a. with the system prior to penetration into the body. The head 10 is the leading component of the spear 7 and the harpoon tether 9 is either in or out of the lumen of spear 7. In FIG. 8b. the harpoon head 10 has been placed through the abdominal wall, through the ePTFE patch 11 and the tether 9 remains in the abdominal wall as a lateral fixation option. The medial side of the patch is indicated by 8 overlying the inferior epigastric vessels 19. The spear carrier is now empty 7.

I claim:

1. An apparatus for laparoscopically patching hernia defects comprising:
a frame of radiating arms or concentric circles of metal or plastic members supporting a fabric patch;
folded into a hollow cannula having a distal end and a proximal end in such a way as to permit delivery into the body resulting in patch placement in planar fashion in the peritoneal space, pleural space, or premembranous extraperitoneal space in a defect or weakness;
the hooked support members hold the fabric and strengthen the area in such a way as to restore the integrity of the wall;
the hooks engage the abdominal wall and eliminate movement between the apparatus and the abdominal wall.

2. The apparatus in claim 1 wherein the stiffening radial or concentric frame is formed with a metal such as super elastic nitinol or firm plastic material fused, glued, or in another way attached, partially or completely to the planar ePTFE or planar fabric portion or planar silicone portion.

3. The prosthesis in claim 1 where the metal frame in claim 2 has multidirectional hooks of sufficient length, directed toward peritoneum to anchor the peritoneum and prevent patch displacement or peritoneal movement.

4. The prosthesis in claim 1 wherein the center of the patch has an anchor cord or fabric element to prevent migration of the patch in the intraperitoneal space, which when pulled up against the abdominal wall will pull the nitinol hooks to engage into the peritoneum forming a prosthesis peritoneal complex with firm resistance to deformation or migration.

5. The prosthesis in claim 1 has a planar shape or preferably a conformal centrally depressed or concave shape to seat in the hernial depression;
and can be also symmetrically depressed medially and laterally being somewhat symmetrical to enable use of the inferior epigastric vessel leash as a supportive point.

6. The prosthesis in claim 1 where the plastic may be an ePTFE fabric or other adhesion resistant fabric such as silicon to resist adhesion formation on the visceral surfaces and has an adherent layer on the side facing the peritoneum fused or attached to the ePTFE or silicon type fabric.

7. The prosthesis in claim 1 which has the entire structure of the device herein described manufactured by injection molding or some similar process to reduce cost;
to create all of the hook elements, webs , anchors, and ribs of a single plastic material that is unitary in construction and is flexible in its application to the tissues.

8. The prosthesis in claim 1. which has preformed holes placed laterally and medially through the planar fabric that may be used from time to time to permit transit of the detachable head of the harpoon fixation device in claim 9 which is passed through the holes in the patch.

9. The method wherein the perihernial area is visualized with an endoscope;
to diagnose the hernia type, to assure the safe position of bowel;
to define the delivery cannula path through the center of the defect;
and allow safe division of the peritoneal sac if required.

10. The method in claim 9 may require a cannula and piercing trocar in claim 1 to create a passage and deliver the mesh complex through the cannula lumen to the repair site directly from the skin through the internal peritoneal hernial depression using a fitted pusher element that forces the patch out maintaining CO2 pneumoperitoneum pressure during this process.

11. The cannula and pusher in claim 10 to be constructed from tubing preferably bending to permit manouver of the cannula without restriction from the thigh anatomy and accurately deliver the patch in claim 1.

12. The method in claim 9 modified allows direct delivery of the patch into the peritoneal space without aid of a cannula.

13. The method in claim 9 can use a harpoon delivered toggle fixation ligature comprising:
a hollow or solid shaft of sufficient strength to carry through the skin and abdominal wall to the peritoneum;
a detachable toggle structure anchored to a ligature that is used from time to time to provide fixation of the hernia patch in claim 1 to the abdominal wall.
